(19) United States
(12) Patent Application Publication
(10) Pub. No.: US 2011/0281343 A1
(43) Pub. Date: Nov. 17, 2011

(54) BIOREACTOR WITH RODS ARRAYED FOR CULTURING ANCHORAGE-DEPENDENT CELLS

(76) Inventor: Roger J. Gay, Belmont, MA (US)

(21) Appl. No.: 13/122,315

(22) PCT Filed: Oct. 2, 2009
(86) PCT No.: PCT/US09/59391

§ 371 Date: Jul. 19, 2011

Related U.S. Application Data
(60) Provisional application No. 61/194,966, filed on Oct. 2, 2008.

(51) Int. Cl. C12M 3/00; C12M 1/34; C12M 1/24

(52) U.S. Cl. 435/287.1; 435/289.1; 435/304.1

(57) ABSTRACT

The instant invention employs an array of rods packed into a suitable chamber to support the culture, e.g., maintenance and/or growth of anchorage-dependent cells that effectively increases the surface area available for cell culture. Adherent cells are seeded and propagated on the surface of the rods. Each rod is accommodated with spacer devices that serve to make it immobile and ensure uniform access to liquid growth medium while minimizing abrasive damage to shear-sensitive cells. Spacer devices of various designs allow the rods to be packed and anchored into bioreactors, e.g., perfusion chambers with flow-through ports or into closed roller bottle-type chambers. Simple monomeric designs of the rods and spacer devices allow for relative ease of manufacture and assembly including solid or hollow rods or supports constructed of fibers or strings consisting of flexible tissue culture treated material that is held fast by threading or knotting between suitable spacer devices with eyelets, holes or other anchoring fixtures. In some embodiments, the rods are composed of tissue culture-treated plastic packed into culture chambers as prepackaged, sterile, disposable single use items.

### RELATED APPLICATIONS

This application claims priority to U.S. Provisional Application No. 61/194,966, filed Oct. 2, 2008, the contents of which are hereby incorporated by reference.

### BACKGROUND OF THE INVENTION

The propagation of anchorage dependent cells requires cellular attachment to a suitable solid support. In the research laboratory, the most common vessels for propagating anchorage dependent cells are sterile, disposable polystyrene tissue culture flasks whose growth surface has been treated to obtain the appropriate charge density for promoting cell adhesion. For scale-up operations, multiple larger flasks or flasks with multiple tiers are typically used. For commercial production of medical products, the use of relatively inexpensive, disposable culture systems offers significant advantages. The use of prepackaged sterile disposable culture vessel precludes potential cross contamination by residuals from previous lots that may result from the reuse of culture vessels or bioreactor components. In addition, the use of disposable culture systems eliminates the need to validate cleaning and sanitization procedures required to qualify the use of recycled components.

Other approaches for larger scale growth of anchorage dependent cells include various types of microcarrier beads and fixed bed culture systems such as porous glass beads and backed glass fibers. Typically, these systems require reaction chambers that are sanitized and sterilized between uses. These systems are not suitable for all cell types, especially shear-sensitive cells.

Current culture vessels and bioreactors used for propagating anchorage-dependent cells have drawbacks. For example, the use of multiple roller bottles and multi-tiered flasks requires multiple individual vessels for large scale production. In addition, the construction and manufacture of self-contained multi-tiered flasks is complex and costly. Maintaining cultures in these flasks is labor intensive requiring medium exchanges of multiple flasks typically performed manually in biological safety cabinets. As with any aseptic operation, the number of open manipulations proportionately increases the risk of microbial contamination.

Compared to stationary tissue culture flasks, microcarrier beads afford an increased surface area for cell growth in relatively smaller vessels. However, the continual stirring required to keep the beads in suspension is not suitable for cells sensitive to the shear forces generated in these systems. Larger scale growth of anchorage dependent cells has also been hampered by inadequate nutrient delivery, particularly limited oxygen diffusion encountered in large culture volumes and at higher cell densities.

Roller bottles are horizontal cylindrical vessels rotating axially that offer advantages including ease of manufacture and good oxygenation properties since the cells alternately spend a potion of their time directly exposed to the atmosphere in the chamber and a portion of their time submerged in cell culture medium. While providing good oxygenation and gentle rolling action suitable for most anchorage-dependent cells, roller bottles occupy significant space relative to the cell growth area provided since cells are grown only on the interior surface of the bottles. As a consequence, roller bottle scale-up and manufacturing operations require large spaces to accommodate multiple roller bottle racks.

To address this limitation, roller bottles with ribs that expand the internal surface area are commercially available. Rippled or pleated roller bottles increase the surface area available for growth by a factor of about two-fold as opposed to roller bottles with smooth-walled interior surfaces.

To further improve efficiency, a number of approaches have been proposed to increase the available surface area in roller bottle-type culture chambers including: inserting a single coil of plastic sheeting in a spiral configuration (U.S. Pat. No. 3,941,661), inserting a sleeve or a liner (U.S. Pat. No. 4,912,058), corrugating the inside growth surface (U.S. Pat. No. 4,824,787), texturing, cratering or multi-cupellating growth surfaces (U.S. Pat. No. 6,130,080), inserting one or more annular rods forming a series of inner concentrically arranged hollow tubes (U.S. Pat. No. 4,317,886), altering cross-sectional shape (U.S. Pat. No. 5,866,419) and corrugating and expanding roller bottles (US Patent Application 20070224679).

One such system has been proposed by Yoo et al., 2002 (U.S. Pat. No. 6,492,163). The Yoo system is composed of a series of hollow tubes with two openings allowing culture medium to enter and exit each tube. In one application, a multitude of cell culture tube bundles are axially arranged around a central rotating shaft within a cylindrical housing assembled in a plurality of roller drums.

Ensuring uniform culture medium circulation, nutrient delivery, aeration, and an otherwise uniform environment for cells in all areas of the culture system is challenging. For example, the Yoo system contains subchambers with multiple hollow tubes. Each tube is designed with holes allowing medium to enter and exit during each rotation cycle. These openings can easily become blocked or otherwise occluded with air bubbles preventing medium access to the cells and disrupting uniformity of flow. In addition, even if effective for culturing anchorage-dependent cells, systems with complex design and construction requiring extensive development, engineering, and manufacturing efforts may hamper commercialization.

The present invention discloses simple monomeric designs and detailed descriptions for constructing easy-to-manufacture bioreactors with increased surface area for cell growth to realize enhanced yields.

### SUMMARY OF THE INVENTION

It is the object of this invention to provide an apparatus of simple design that enables the culture (e.g., growth and/or maintenance) of anchorage dependent cells in an easy to manufacture and easy to use configuration. The present invention overcomes the limitations associated with the use of multiple tissue culture flasks use by greatly increasing the surface area available for cell growth in bioreactors of moderate dimensions. In addition, current art procedures, conditions, and bioreactors for culturing cells can be readily adapted to embodiments of the disclosed invention.

It is another object of the present invention to provide simple monomeric designs allowing ease of construction and assembly.

In accordance with an aspect of the invention, there is provided an array of solid or flexible rods (e.g., rigid rods, taught fibers, woven fibers) that allows for the seeding, propagation and harvest of anchorage dependent cells within a bioreactor housing or culture vessel.

In one embodiment, the rods are positioned and anchored within the chamber with at least one spacer device affixed to each rod either as a separate component, as an integral part of the rod design, as part of the culture chamber, or as an insert placed in the chamber (such as a disc or a plate with holes) at the center or at one or both ends of the chamber). The spacer device(s) serves to anchor the rods to prevent movement relative to other rods during culture. In one embodiment, the spacer device keeps each rod from coming into contact with neighboring rods. In another embodiment, rods may be in contact with each other, but do not move during culture of the cells.

In one embodiment, a bioreactor of the invention comprises at least one spacer device. In a preferred embodiment, a bioreactor of the invention comprises two spacer devices. In one embodiment, additional spacer devices may be present to further stabilize the rods in the bioreactor if desired.

The array of rods presents a growth surface for anchorage dependent cells and provides uniform and sufficient access to liquid nutrient medium. The rods may be solid or hollow. The rods may be rigid or composed of a flexible material such as a fiber, string or thread stretched taut between anchoring plates effectively forming a rigid support. The rods may be of a porous or non-porous material. In one embodiment, the rods are solid so that the cells may only attach to the external surface of the rods and uniformity of exposure to the media is ensured. Cells attached to the anchored rods are protected from shear forces and abrasion since the rods do not move with respect to each other during culture.

In one embodiment, the bioreactor chamber is a closed roller bottle-type system into which the rods and one or more spacer devices are packed and used in a manner similar to traditional roller bottle cultures.

In another embodiment, the bioreactor chamber is placed in a perfusion loop with pumps allowing for constant or pulsatile medium exchange and for a convenient way to seed and harvest cells from the bioreactor. Such a bioreactor may comprise holes for easy exchange of medium. The perfusion system simplifies medium changes and cell harvesting in that most operations can be carried out in a closed loop with sterile quick-connects for changing medium reservoirs thereby reducing contamination and ensuring operator safety.

The present invention also allows for the scale-up of shear sensitive anchorage dependent cells in relatively inexpensive, disposable cell culture vessels. By greatly increasing the surface available for cell growth, the invention described herein provides a design that reduces the number of vessels needed when compared to conventional cell culture flasks, including traditional roller bottles and flasks with multiple horizontal tiered surfaces. Collectively, the invention allows for significant savings in labor, reduction in facility size, and minimizes the risks of microbial contamination by reducing the number of culture chambers required. The components of this system are designed to be relatively inexpensive to manufacture using current culture plastic-ware materials and manufacturing processes. The system is also designed to be readily adaptable for culturing anchorage cells using standard current art methods and procedures.

The instant invention is based, inter alia, on the discovery that a simple component geometry arranged in an array provides a support structure for the propagation of anchorage dependent cells. One advantage of the monomeric construction approach is the simplicity of manufacture. The spacer devices that segregate and align the rods are likewise simple in design and relatively inexpensive to manufacture and assemble.

Essential elements of the invention include an array of rods arrayed in a cell culture chamber. In one embodiment, the rods are arrayed in parallel. In another embodiment, at least one rod is not parallel to at least one other rod in the array. For example, rods in the form of flexible fibers may be arrayed between two spacer devices in a pattern that does not result in a parallel arrangement of the fiberous rods or which comprises at least one rod which is not arranged parallel to at least one other rod, e.g., is at an angle relative to a spacer device.

As used herein, the term “rod” includes rigid rods as well as rods composed of flexible fibers (including those comprised of tissue culture-treated plastic or other surface treated flexible fibers) stretched taut between spacer devices (e.g., plates or other anchoring devices) effectively forming rigid rods when under tension. In another embodiment, at least one rod may be of more complex geometry, e.g., a complex rod made from fiberous plastic fibers woven together.

With respect to the dimensions of rods, they may range in diameter from approximately (+/−0.01 mm) 0.01 mm to approximately 10000 mm (10 cm) in diameter (e.g., 0.01, 0.03, 0.1, 0.3, 1.0, 3.0, 10, 30, 100, 300, 1000, 3000, to 10000 mm).

The growth surface of each rod is cell-culture appropriate (e.g., naturally or after treating) providing an optimal substratum for cell adhesion and growth. The system provides a large surface area available for cell growth in a relatively small vessel making it easier and practical for use, compared to using multiple conventional tissue culture vessels.

Exemplary materials from which rods can be made include glass, rigid plastic (e.g., polypropylene or polystyrene), fiber, flexible glass, and flexible plastic. In one embodiment, a single spool of fiber capable of supporting cell growth is attached (e.g., loomed, stitched or knotted) to eyelets, knobs or other fasteners at opposing ends of the reactor such that complicated arrays are fabricated without the need for glue or other fasteners. The diameter and cross-sectional area of the fiber could be varied as could the geometry of the loomed configuration.

In some embodiments, all components are molded and assembled prior to insertion into a pre-existing culture chamber and sterilization.

The design features outlined below in embodiment “A” provide a means to greatly increase the surface available for cell growth using a parallel array of rods with spacer devices packed into a conventional roller bottle.

The design features outlined below in embodiment “B” provide a means to greatly increase the surface available for cell growth using a parallel array of rods with spacer devices packed into a perfusion chamber allowing inflow and egress of nutrient medium.

The features outlined for exemplary embodiments “A” and “B” also provide for using a parallel array of rods with spacer devices packed into a hybrid chamber incorporating aspects of a roller bottle and of a perfusion-type of bioreactor during different phases of cell growth and for different cell culture manipulations. For example, cells could be seeded in the chamber while rotating on a roller drum, with medium changes and cell propagation occurring in the perfusion mode and final cell harvest performed as a roller bottle.

### BRIEF DESCRIPTION OF THE DRAWINGS

FIG. 1 shows a side view of a conventional roller bottle as an exemplary culture chamber.

FIG. 2 shows a roller bottle packed with a parallel array of rods in an annular configuration lining the perimeter of the chamber. The arrangement creates a central cylindrically shaped area of void volume extending the length of the chamber.

FIG. 3 shows a top-down perspective view of a roller bottle drawn to actual scale. The dashed lines delineate nine quadrants each encompassing a 40 degree arc. Smaller circles in one of the quadrants represent a head-on view of rods packed in the chamber.

FIG. 4 shows a side view of a rod drawn to actual scale.

FIG. 5 shows a side view of a rod with knobs at both ends, drawn to actual scale.

FIG. 6 outlines the shape of a single rod-packet positioned within a roller bottle.

FIG. 7 shows the two opposing spacer devices of a rod-packet placed within a roller bottle. For the sake of clarity, only one of the 41 rods and the corresponding 41 holes in each spacer is illustrated in the drawing.

FIG. 8 shows a head-on view of the spacer device with 41 uniformly spaced holes to anchor the rods in a rod-packet.

FIG. 9 shows an off-side view of a spacer device revealing a groove in the canted underside surface. The surface with the groove faces the interior void volume of the roller bottle. The canted surface conforms to the vertical rise of the mound when placed at the base of the roller bottle. The groove in the canted surface engages a retaining device when placed at the top of the roller bottle. The surface with the register of holes is facing down and not visible from this perspective.

FIG. 10 shows a top view of a roller bottle with nine spacer devices anchored in place with an O-ring engaging the grooves in the abutting spacer devices that surround the opening of the roller bottle.

FIG. 11 shows a rod with knobs at both ends.

FIG. 12 shows a head-on view of a representative section of a stack of knobbed rods.

FIG. 13 shows a side view of a rod-packet containing knobbed rods packed in a staggered array to accommodate the internal contours of the vertical rise of the mound at the base of the roller bottle.

FIG. 14 shows a head-on view of a representative section of a parallel array of uniformly spaced rods with flow-through spacer devices adapted for use in a perfusion chamber.

FIG. 15 shows a head-on view of a spacer device with a ridged hole. Note that ridged holes may be a feature of individual collars inserted over both ends of each rod or they may be part of a spacer device containing a register of ridged holes.

FIG. 16 shows a side view of a spacer device with a ridged hole.

FIG. 17 shows a smooth rod. The arrow denotes insertion of a smooth rod into a spacer device with a ridged hole.

FIG. 18 shows a head-on view of a spacer device with a smooth bore hole. Note that smooth holes may be a feature of individual collars inserted over both ends of each finned rod or they may be part of a spacer device containing a register of smooth holes.

FIG. 19 shows a side view of a spacer device with a smooth hole.

FIG. 20 shows a rod with fins on both ends. The arrow denotes insertion of a finned rod into a spacer device with a smooth bore hole.

### DETAILED DESCRIPTION

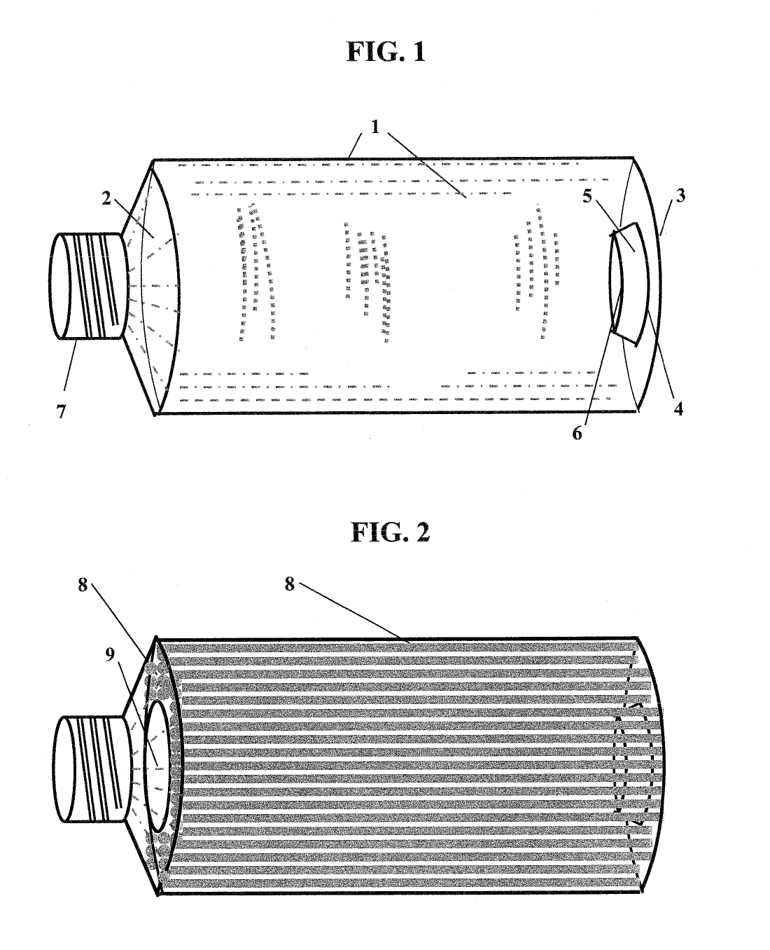

An exemplary embodiment of the invention is set forth in FIG. 1. With reference to FIG. 1 a conventional plastic roller bottle with approximate dimensions as follows: wall (1), length 233 mm from the base (3) to the lowest portion of the top (2), chamber inner diameter 113 mm, threaded opening (7) with an internal diameter of 42 mm that accommodates a screw-on cap is packed with an array of parallel rods (FIG. 2). The rods are composed of tissue culture appropriate material (e.g., treated plastic) to promote cell adhesion and growth. In this example, the dimensions of each rod (FIG. 4) are approximately 232 mm in total length and 3 mm in diameter along the length of the shaft. The rods may have a smooth or corrugated surface. For one mode of assembly and spacing, the rods have knobs (11) at both ends approximately 4 mm in diameter (FIG. 5).

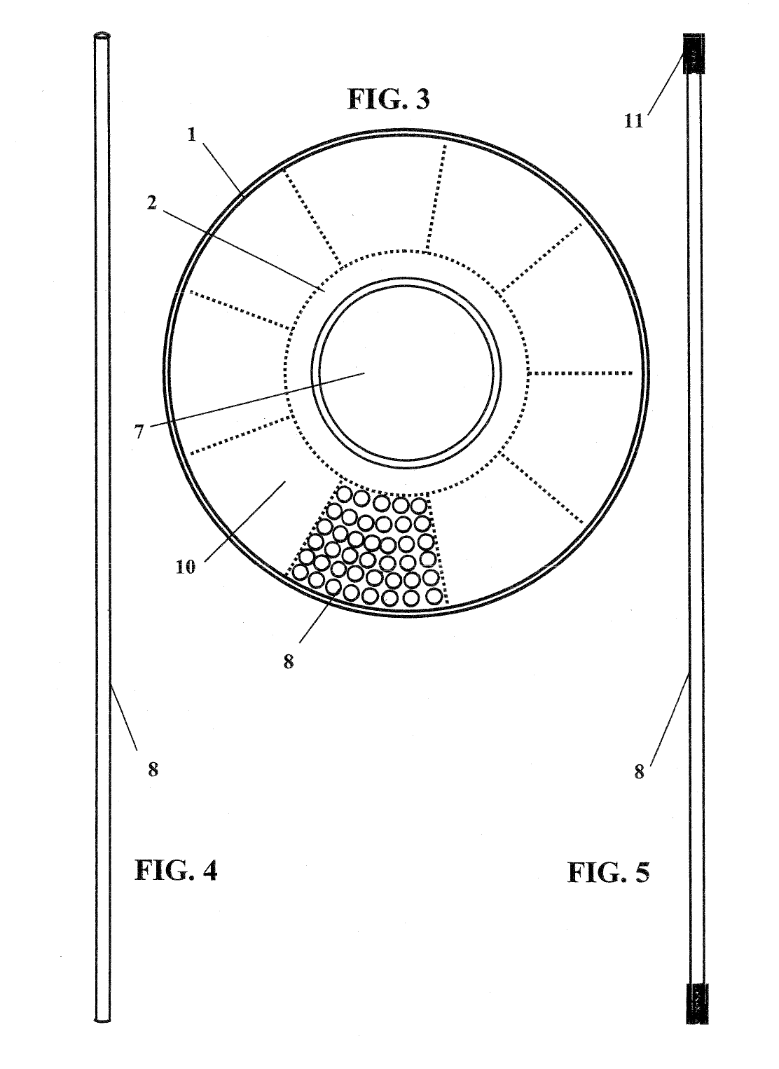

The rods run parallel to the wall of the roller bottle and are annularly arranged along the wall stacked approximately 27 mm in height extending from the wall to the interior of the roller bottle (FIG. 2). The external surface of each rod (8) is uniformly spaced approximately 1 mm from each of its nearest neighbors along the entire length of the rod (FIGS. 2 and 3). The packets of rods are anchored in place within the roller bottle by one of several means. Each packet is composed of 41 rods (8) (FIG. 3) stacked in a shape resembling an isosceles trapezoid (10) when viewed on end (FIG. 3) but with a curved base conforming to the arc of the roller bottle's wall.

A total of nine packets are inserted such that the entire perimeter of the inner wall of the roller bottle is lined with rods creating a central cylindrical core of empty space (9) (FIG. 2) running the length of roller bottle approximately 60 mm in diameter.

When packed in parallel array, each roller bottle can accommodate 9 packets containing a total of 369 rods with an exposed length of 226 mm available for cell growth on the external surface. The collective surface area of all the rods in a roller bottle is approximately 7,860 cm².

(3.14×3mm)×(226mm)=21.3cm²/rod

9packets/roller bottle×41rods/packet×21.3cm²/rod=7,860cm²

If the interior wall of the roller bottle wall is cell culture-treated, the growth area of a smooth-walled roller bottle of these dimensions would be increased from 850 cm² to a total of 8710 cm² or by a factor of greater than 10, i.e., 10.2-fold. Therefore, in one embodiment, a bioreactor of the invention has a growth area at least about 10 fold greater than a bioreactor that does not comprise the array of rods.

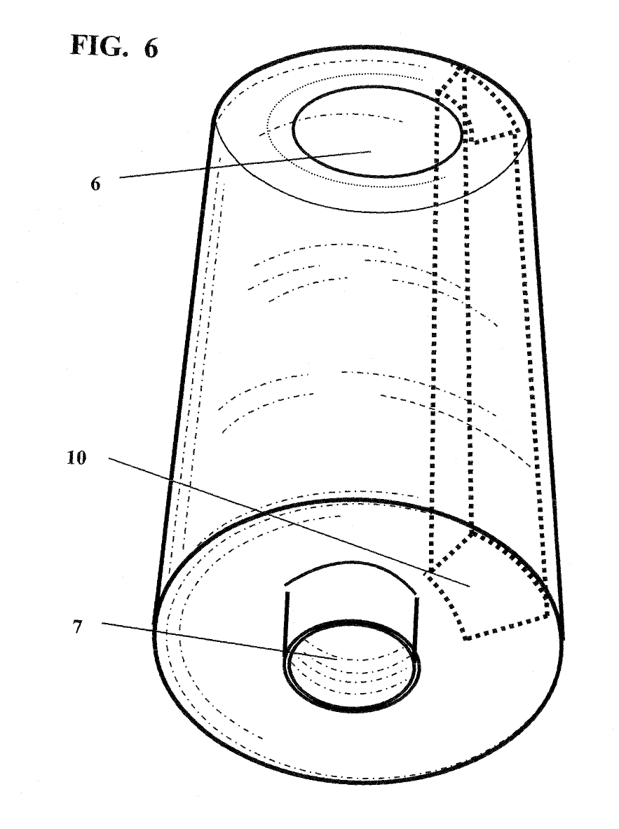

The general shape (10) occupied by a single rod-packet within a roller bottle is shown in FIG. 6 denoted by the dotted lines. Several simple spacing and anchoring devices to align the rods are suitable for this embodiment. These schemes involve the preassembly of rod-packets of the general shape and geometry shown in FIG. 6, subsequent insertion of the packets though the opening (7) of the roller bottle, and anchoring along the perimeter of the inner wall of the roller bottle. Several such spacing devices and assembly schemes are described below.

### Design and Assembly in Roller Bottle Example 1

(Rods Inserted into Spacer Devices with Holes in Register)

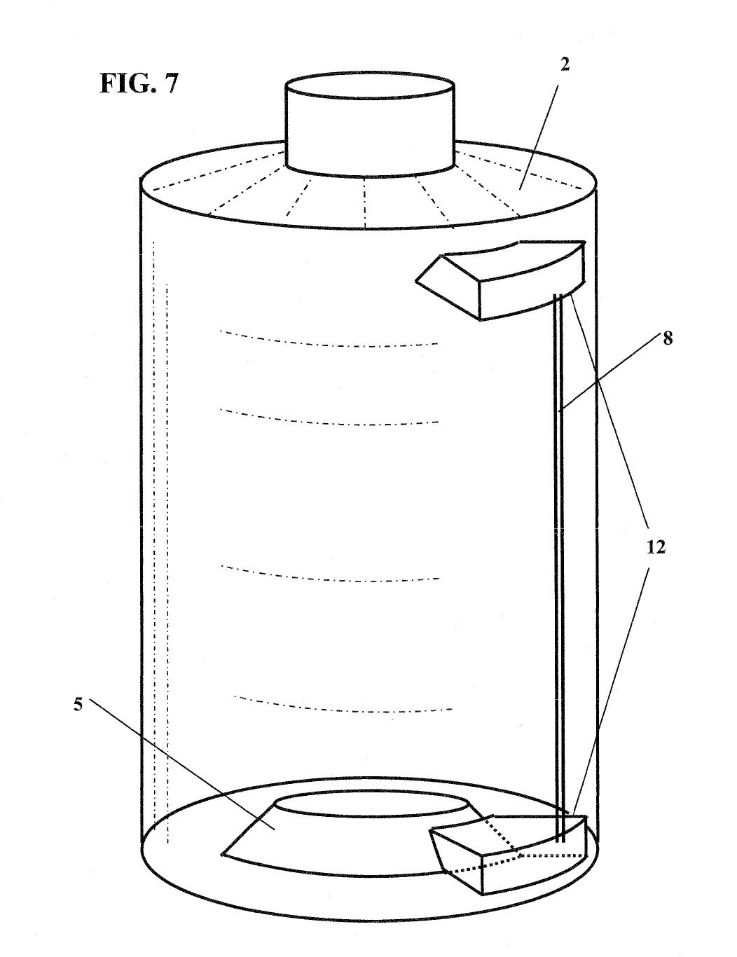

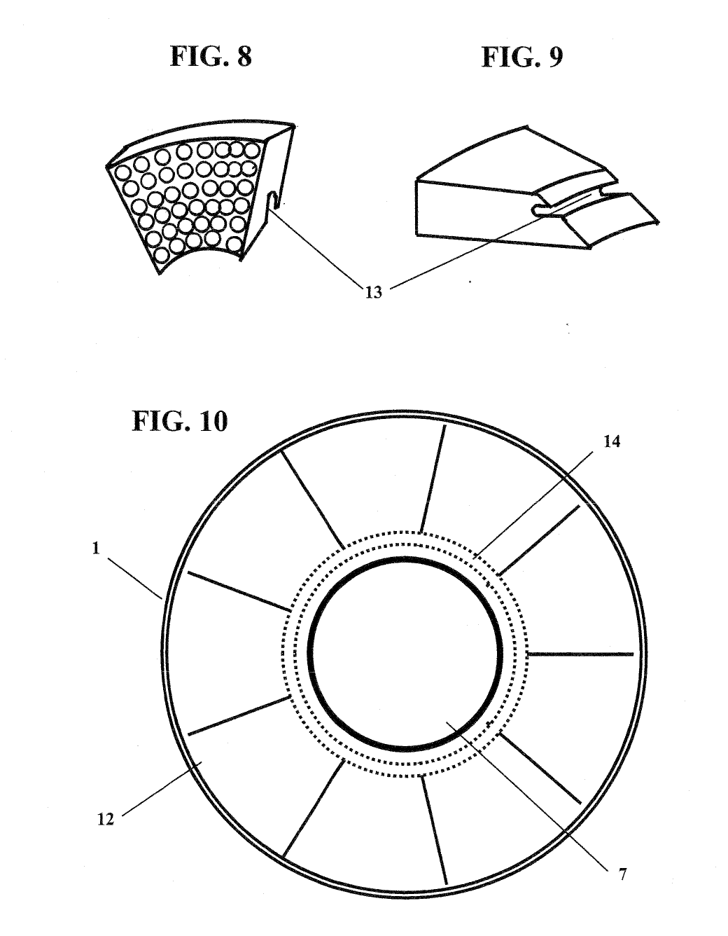

Rods (8) (FIG. 4) are composed of tissue culture-treated polystyrene or other suitable material approximately 233 mm in total length and 3 mm in diameter. Spacer devices (12) (FIG. 7) each with one surface containing 41 uniformly spaced holes to accommodate 41 rods (8) (FIG. 3) is formed from tissue-culture grade plastic or other suitable material. Holes are designed to engage and anchor the 3 mm diameter rods, are 1 to 3 mm in depth, and approximately spaced 1 mm from all neighboring holes (FIG. 8). The ends of the rods are inserted into one of two register plates (two spacing device for each packet of rods) (FIG. 7). For each rod-packet, 41 rods are suspended between two facing spacer devices. Each of nine preassembled packets of rods with register plates at both ends are then placed into the roller bottle. One of the spacer devices is positioned at the base (3) and the other at another at the top (2) of the roller bottle (FIG. 7).

The surface of the spacer device facing the internal void volume (9) of the roller bottle is shaped to conform to the vertical rise (5) (FIG. 7) of the mound at the base of the roller bottle and is grooved (13) to accommodate a retaining device at the top of the roller bottle (FIGS. 8 and 9). When all nine packets are inserted they are anchored in place at the base. An O-ring (14) or other retaining device (FIG. 10) composed of suitable tissue culture grade material such as flexible tissue culture-grade tubing is then inserted into the roller bottle. The retaining device engages the grooves on the top of the spacer devices closest to the top of the roller bottle. The retaining device exerts outward pressure and ensures that the rods are pressing against the wall of the roller bottle.

### Design and Assembly in Roller Bottle Example 2

(Stacks of Rods with Knobbed Ends)

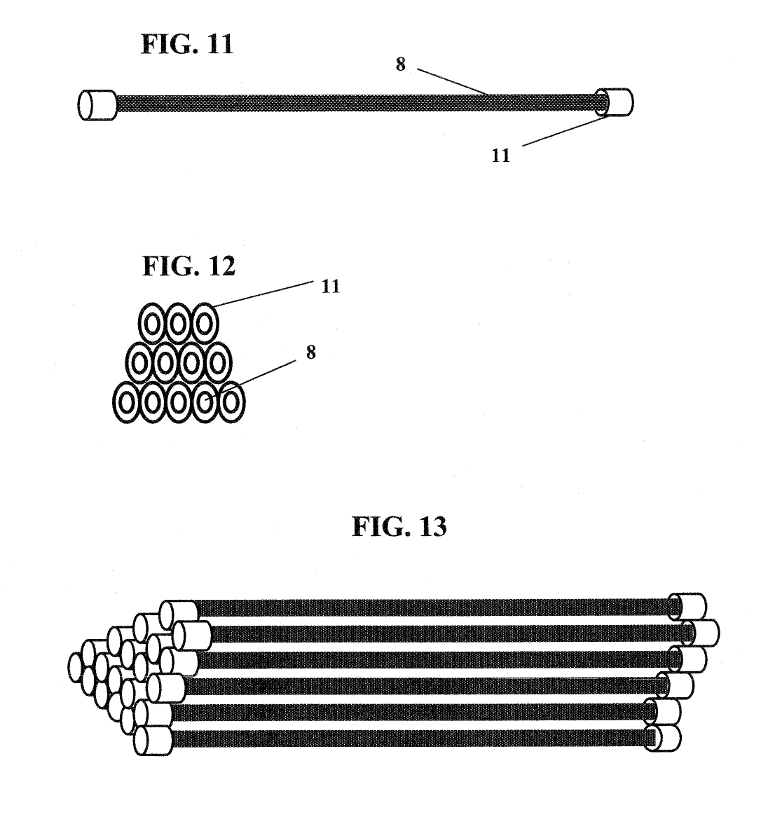

Rods are composed of tissue culture-treated polystyrene or other suitable material approximately 232 mm in total length (222 mm available for cell attachment and growth) and 3 mm in diameter (FIG. 5). Both ends of each rod have cylindrical knobs 11 (FIG. 11) approximately 5 mm in length and 4 mm in diameter FIG. 5). Knobs can be an integral part of the rod formed at the time of casting or may be affixed to the rods by capping with a suitable material such as ensheathment with cut sections of flexible tissue culture-grade tubing. The knobbed rods are stacked in parallel array of 41 rods per packet forming the general shape outlined in FIG. 13 forming the uniform representative geometry shown in FIG. 12. The rods are stacked in a staggered manner to ensure that the packets conform to and are thus anchored against the mound (5) at the base of the roller bottle (FIG. 13). The stacked packets are bound, banded, glued or otherwise affixed together to maintain the appropriate geometry conforming to the contours of the roller bottle. Nine preassembled packets may be inserted into the roller bottle with one end of each packet contacting the mound at the base of the roller bottle thereby anchoring the packet in position at the base. An O-ring (14) or other retaining device composed of suitable tissue culture grade material such as flexible tissue culture-grade tubing is then inserted into the roller bottle. The retaining device contacts the knobs facing the top of the roller bottle similar to the arrangement shown in FIG. 10 where the O-ring engages a spacer device. The retaining device exerts outward pressure and ensuring that the rods are pressing against the wall of the roller bottle.

Another type of spacer device is a donut-shaped register with uniformly spaced holes constructed of flexible tissue culture-grade plastic or rubber allowing the spacer to be folded for insertion through the opening in the roller bottle. In this case, the spacer device is inserted into the roller bottle and positioned at the base of the roller bottle surrounding the mound. Rods are then inserted into the holes in the register plate. A retaining device, such as those mentioned above, secures the knobbed ends of the rods facing the top of the roller bottle.

Another assembly employs a flexible tissue culture treated plastic or other suitable material wherein the starting material is a spool of fiber or string that is threaded, knotted or woven between two or more spacer devices fitted with holes, pegs or eyelets and stretched taut effectively forming rods under tension; simulating the design outlined above.

After assembly, the roller bottle containing the anchored rod packets may be capped, bagged and sterilized.

The designs and assembly procedures disclosed above can be adapted to other chambers such as larger roller bottles and cylindrical carboys. For example, a 20 liter screw-capped carboy tipped on its side has the approximate dimensions of: 350 mm straight wall length, an internal diameter of 265 mm, and an opening with an internal diameter of about 80 mm. A chamber of this size can accommodate 16 rod-packets aligned on the perimeter of the chamber with each packet containing approximately 65 rods in a stack approximately 75 mm tall and about 50 mm at the base of each packet. Each rod is approximately 350 mm in total length, with 344 mm of length available for growth, 3 mm in diameter, with a uniform spacing of 2 mm between the growth surface of each rod and its nearest neighbor.

The total surface available for growth in a 20 L carboy packed with 1040 rods is:

(3.14×3mm)×(344mm)=32.4cm²/rod

16packets/roller bottle×65rods/packet×32.4cm²/rod=33,696cm²

or the equivalent of forty 850 cm² roller bottles.

It will be understood that these calculations are based on the use of existing roller bottles or carboys and that vesicles of unique size may also be used ant the rods may be made to fit appropriately.

In one embodiment, to initiate cultures, cell suspensions are introduced into the roller bottle chamber packed with rods. The number of cells seeded should be adjusted accordingly to compensate for the increased available surface area. The volume of cell suspension added may be similar to a comparable sized roller bottle, typically about one third of the total volume and sufficient to completely submerge the rods on the bottom of the rotation cycle. Medium is added to the roller bottle to a depth of approximately 3 cm immersing all packets containing rods during the course of each rotation while maintaining a medium level approximately 1 cm below the opening of the roller bottle.

To promote more rapid and uniform cell attachment, the volume of cell the suspension used for seeding may be increased above the opening provided a solid cap is used. For this procedure, it may also be possible to insert a closed-ended displacement cylinder designed to displace medium from the central void volume. In this way cells are restricted to the perimeter of the roller where the rods are positioned. The roller bottle is placed on a roller bottle apparatus using conditions optimal for the plating efficiency of the particular cell type. During cell seeding, rotation speeds are typically set lower (0.1 to 1 RPM) than during the growth phase and rotations may be intermittent to allow the cells to attach without undue agitation. After cell attachment is complete, the displacement cylinder (if any) is removed and any excess medium is discarded. The medium level should be sufficient cover the rods at the bottom of the rotation cycle but below the roller bottle opening if a gas-permeable cap is used to allow for free gas exchange (typically carbon dioxide to maintain pH in an bicarbonate based buffered medium). Medium changes are more frequent than those required for conventional roller bottles and should be adjusted accordingly to account the increased surface area directly proportional to the number of cells. In one embodiment, a break-away rod, i.e., one or more rods that are not part of a fixed assembly, may be included that may be easily removed during cell culture. Cell growth, morphology and other properties are assessed during growth in culture by aseptic removal of single break-away rods using sterile forceps to grasp and remove a representative rod. Such a rod may be fitted with a means for grasping, e.g., a loop or special end). The most readily accessible rods removed for monitoring are those closest to central core of the roller bottle. In one embodiment, more than one break-away rod may be included. Cells can be examined directly while attached to the rod under an inverted light microscope. Rods removed for monitoring can also be placed in a dish or tube where adhering cells may be trypsinized to assess cell number, viability, or other cell attributes using standard cell culture methodologies.

If treated to promote cell attachment, the walls of the roller bottle may also serve as a cell attachment and growth surfaces.

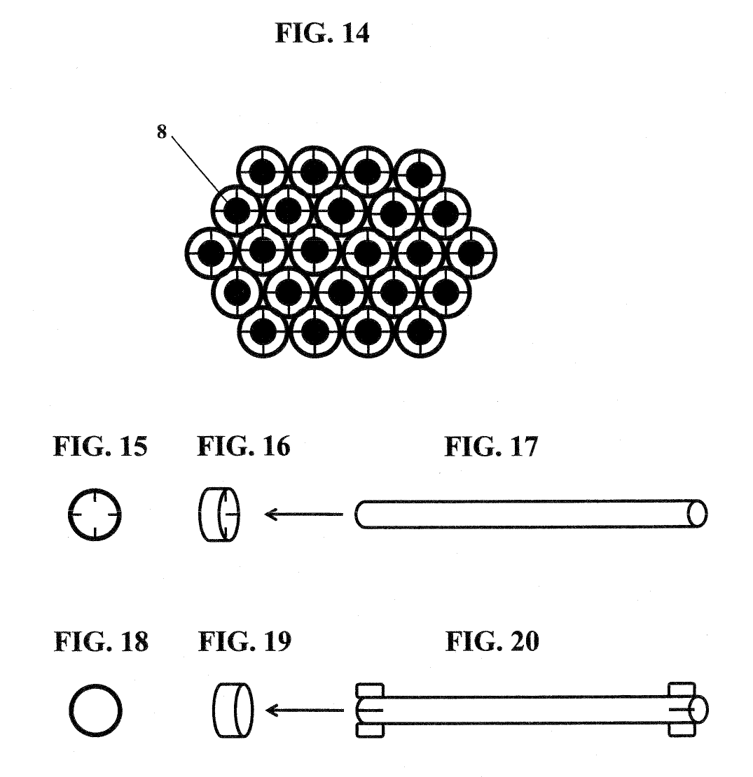

In another embodiment, a bioreactor of the invention consists of a cylindrical perfusion chamber with ports at the top and the base for allowing continuous or pulsatile circulation including inflow, and egress of liquid medium, cells or cellular products. One or more ports at opposing ends of the cylinder are composed of hollow posts to accommodate tubing of varying diameters with a typical diameter inner diameter in the range of 1 cm. The chamber is composed of tissue culture grade plastic with the dimensions approximating those of a 20 liter carboy tipped on its side with 350 mm straight wall length, an internal diameter of 265 mm, and an opening with an internal diameter of about 80 mm. In this embodiment, the ends of the rods are and spacer devices are designed to allow for the uniform and free flow of liquid culture medium along the length of each rod. The geometry of this design is shown in FIG. 14.

A chamber of this size can accommodate 16 rod-packets aligned on the perimeter of the chamber with each packet containing approximately 65 rods in a stack approximately 75 mm tall and about 50 mm at the base of each packet. Each rod is approximately 350 mm in total length, with 344 mm of length available for growth, 3 mm in diameter, with a uniform spacing of 2 mm between the growth surface of each rod and its nearest neighbor.

After insertion of the 16 rod-packets at the perimeter of the chamber, a central cylindrically shaped rod-packet is then inserted to occupy the central core of the chamber such that the entire chamber is packed with a parallel array of rods. The central cylindrical rod-packet is composed of approximately 150 rods approximately 75 mm in diameter of same flow-through design depicted in FIG. 14.

The surface available for growth in chamber with dimensions approximating a 20 L carboy packed with 16 rod-packets (65 rods per packet) at the perimeter is 33,696 cm² (see previous section for details of calculation) and packet at the central cylindrical core is

150rods/packet)×32.4cm²/rod=4,860cm²

for a total of 38,566 cm² for the bioreactor or the equivalent of forty five, 850 cm² roller bottles.

The fabrication methods and general geometry of rod-packets, spacer devices, and their arrangement and assembly within the culture chamber are analogous to those described in Embodiment A. However, in this instance, the rods and spacer devices are of a flow-through design and the final insertion of a cylindrical shaped rod-packet occupies the internal core of the chamber. As in the examples of assembly 1 and 2 for embodiment A, the spacer device may be a register plates with holes at opposing ends of the rods or the rods themselves may be fitted with end-collars to ensure uniform spacing.

To manufacture this geometry several methods are disclosed below.

### Design and Assembly in a Perfusion Chamber Example 1

(Rods Inserted into Spacer Devices with Holes in Register)

Spacer devices with panels of uniformly spaced holes may be comprised of ridged holes to accommodate smooth rods (FIGS. 15, 16, 17) or rods with fins or ribs on both ends inserted into smooth holes (FIGS. 18, 19, 20). The assembly and general shape of the rod packets and spacer devices positioned at the perimeter of the chamber are similar to that shown in FIGS. 7, 8, 9, 10 with the flow-through design shown in FIG. 14. For the rod packet occupying the central core the shape is cylindrical.

### Design and Assembly in a Perfusion Chamber Example 2

(Stacks of Rods with Flow-Through Collars)

For this construction method, both ends of each rod are fitted with an appropriate collar. For smooth rods, both ends of each rod are fitted with ribbed collars (FIGS. 15,16). Rod packets positioned at the perimeter of the chamber are similar in general shape to that shown in FIG. 13 with the flow-through design shown in FIG. 14. For the rod packet occupying the central core the shape is cylindrical. The stacked packets are bound, banded, glued or otherwise affixed together to maintain the appropriate geometry conforming to the contours of the culture chamber.

The above design configurations for a perfusion chamber can also be adapted to incorporate tubes as rods.

Depending on the cell type and density, the system may require additional oxygenation to maintain optimal cell growth and maintenance. In this case, the array may consist of a coaxial system wherein fibers or tubes carrying oxygen, carbon dioxide and other gases will be interspersed within the rods containing cells.

The chamber and perfusion lines can be equipped with various sensors (oxygen, lactic acid, glucose, ammonia, pH, etc.) to monitor cellular metabolism and these sensors can be interfaced with control systems such as oxygen sparging and medium pumps to optimally control the rate and duration of nutrient delivery.

The reaction chamber, with ports, grids, rods and spacers may be assembled and sterilized by the manufacturer and shipped ready for use.

What is claimed is:

1. A bioreactor for the culture of cells comprising a culture chamber encasing an array of rods that allows for the seeding, propagation and harvest of anchorage dependent cells or their products, the array of rods comprising surfaces that promote cellular attachment, maintenance or growth of anchorage dependent cells within the culture chamber, wherein each rod is affixed at least one point to at least one spacer device, and wherein during culture of cells the rods are immobile and the surfaces of the rods have uniform contact with growth medium.

2. The bioreactor of claim 1, wherein said chamber is a perfusion chamber with ports allowing liquid nutrient medium and cell suspension inflow and removal.

3. The bioreactor of claim 1, wherein said chamber is a roller bottle-type of chamber.

4. The bioreactor of claim 1, wherein the rods are comprised of a rigid material.

5. The bioreactor of claim 4, wherein the rods are arranged in parallel.

6. The bioreactor of claim 1, wherein the rods are comprised of a flexible material stretched taut to form a rod.

7. The bioreactor of claim 1, wherein said chamber is a hybrid with properties of both a roller bottle-type of chamber and a perfusion chamber.

8. The bioreactor of claim 1 wherein said bioreactor comprises two spacer devices which are knobs positioned at one or both ends of the rods.

9. The spacer devices of claim 8 wherein said spacer knobs are composed of flexible tubing or O-rings that ensheath the ends of each rod.

10. The bioreactor of claim 1, wherein said spacer device consists of spikes, ribs or fins either attached as a separate component, or as an integral part of each rod.

11. The bioreactor of claim 1, wherein said spacer device comprises external lateral ribs on the rod to which collars or other spacing components are seated.

12. The bioreactor of claim 1, wherein said rods and said spacer device are of consolidated construction or are initially separate components consolidated into a single unit using fusion methods such as heat, adhesives, tape or other binding elements such as bands or sheathes.

13. The bioreactor of claim 1, wherein said rods and spacer device are fashioned with interlocking features to permit subassembly or final assembly or both.

14. The bioreactor of claim 1, wherein said spacer device comprises a plate with a series of attachment or insertion sites such as holes, notches or spikes, and wherein the plate is positioned in the chamber and each rod is arrayed via affixing to the attachment or insertion sites in the plate.

15. The bioreactor of claim 1, wherein said bioreactor comprises at least two spacer devices which are the of the same type or are of different types.

16. The bioreactor of claim 1, wherein said spacer device contacts the internal contours of said chamber thereby anchoring the rods to the chamber wall and supporting said rods in an array.

17. The bioreactor of claim 1, wherein said rods have a diameter ranging from 0.01 mm to 10 cm and a length ranging from 1 cm to 100 M.

18. The bioreactor of claim 1, wherein said rods have a cross sectional geometry of a circle, square, hexagon, triangle, rectangle or other polygon.

19. The bioreactor of claim 1, wherein said rods are rippled, corrugated or grooved to increase the surface area available for growth

20. The bioreactor of claim 1, wherein said rods are composed of porous materials.

21. The bioreactor of claim 1, wherein said rods are composed of tissue culture grade plastic such as polystyrene, glass, bone, collagen, ceramic, foam or other material suitable for cell growth in culture.

22. The bioreactor of claim 1 wherein the growth surfaces of said rods have been treated to alter electrostatic charge properties such as density and polarity.

23. The bioreactor of claim 1 wherein the growth surfaces of said rods have been treated with biological molecules such as poly-D-lysine, proteins, glycoproteins or other factors to enhance cell adhesion and/or growth.

24. The bioreactor of claim 1, wherein said rods are hollow tubes or fibers which allow cells to attach and grow on the external surface of the rod.

25. The rods of claim 24 where said hollow tubes or fibers are treated to promote the cell attachment and growth on the external surface.

26. The bioreactor of claim 1, wherein said rods are in proximity to or interspersed among hollow fibers or porous tubes or said chamber is equipped with sparging devices or other aeration devices designed to enhance gas exchange and oxygenation to support cell growth.

27. The bioreactor of claim 1, wherein a devise is inserted in the central portion of the culture chamber to keep the rods in parallel at the perimeter of the chamber.

28. The inserted device of claim 27 which comprises holes that allow for free passage of nutrient medium to the cells attached to rods

29. The inserted device of claim 27 that is sealed and excludes medium from the central portion of the bioreactor.

30. The bioreactor of claim 1 wherein said chamber incorporates pumps with tubes or other feeding devices attached to a nutrient medium reservoir to automatically exchange nutrient medium in the bioreactor.

31. The bioreactor of claim 1 that incorporates sensors to monitor cell growth, metabolism or product output.

32. The bioreactor of claim 1 that incorporates control devises for changing the frequency, duration or volume of nutrient medium or gas exchange.

33. The bioreactor of claim 1, wherein at least one of said rods can be removed to facilitate monitoring of cell growth, morphology, metabolism or product yield.
